(19) United States
(12) Patent Application Publication
(10) Pub. No.: US 2006/0030026 A1
(43) Pub. Date: Feb. 9, 2006

(54) Transilluminator having light emitting diode (LED) array

(76) Inventor: Gustavo Garcia, East Setauket, NY (US)

Correspondence Address: Robert E. Cannuscio;Drinker Biddle & Reath LLP, One Logan Square, 18th & Cherry Streets, Philadelphia, PA, 19103-6996, US

(21) Appl. No.: 10/910,032

(22) Filed: Aug. 3, 2004

(51) Int. Cl. C12M 1/34

(52) U.S. Cl. 435/287.1

(57) ABSTRACT

A transilluminator is provided for enhanced visualization of an object/specimen during its examination and/or analysis such as during nucleic acid fragment visualization. In the described embodiment, the transilluminator includes a transmittance plate adapted to support the specimen being tested, and an array of LEDs adapted to emit light through the plate for illumination of the specimen. The transmittance plate, LEDs or a combination thereof filters and/or emits light energy within a predefined spectrum of light energy. In one preferred embodiment, the illuminating light is in the ultraviolet or near-ultraviolet light spectrums to stimulate or fluoresce dyes within the specimens. Furthermore, two or more LEDs which vary in wavelength emission may be employed for the purpose of varying the illumination characteristics of the transilluminator.

### TECHNICAL FIELD

The present invention relates to a transilluminator for visually enhancing objects/specimens during examination, and more particularly, to a transilluminator for electrophoresis apparatus having an array of low-energy, light emitting diodes (LEDs) for producing filtered/controllable light emissions.

### BACKGROUND OF THE INVENTION

Transilluminators function to enhance the visualization of an object/specimen during its examination, evaluation and/or analysis. Such apparatus are routinely employed in lab facilities for providing the requisite backlighting of photographed images, e.g., X-rays, autoradiography. Yet other applications for transilluminators are found in laboratories conducting nucleic acid research and gene identification/codification. One particular application (to which the present invention is directed), relates to optical densitometry wherein nucleic acid fragments are examined and analyzed for various purposes including the science of forensic analysis.

Optical densitometry is a methodology for separating, identifying, visualizing and purifying nucleic acid (DNA, RNA and the like) fragments. The methodology, more commonly known as electrophoresis, uses an electrical charge to separate molecules in a sample (typically placed in a viscous gel). More specifically, nucleic acid fragments to be examined are placed in an electrophoretic gel (e.g., agarose gel) and caused to migrate in “bands” (i.e., in juxtaposed linear paths) through the application of an electrical field. The migration rate of the nucleic acid fragments through the electrophoretic fluid/gel is dependent upon the molecular size of the nucleic acid molecules, the concentration of the electrophoretic fluid/gel, and, of course, the time in, and strength of, the electrical field. After a prescribed period of time, the nucleic acid fragments separate with the gel. The separated fragments are then typically stained with a dye, such as UV and more recently visibly activated dyes, to permit viewing of the separate fragments.

The fragment separation is best visualized/analyzed by placing the nucleic acid specimen in the presence of ultraviolet, and/or near-ultraviolet light using a conventional transilluminator. In the context of a electrophoresis apparatus, a transilluminator is the device which supports and provides the requisite backlighting for examining the stained nucleic acid fragments.

A more thorough understanding of electrophoresis and the various elements/components which are to be combined to produce an electrophoresis device may be had by reference to Smoot et al. U.S. Pat. No. 4,657,655 wherein a transilluminator is combined with a variety of other elements to produce an electrophoresis device. For convenience of description, FIG. 1 thereof has been reproduced and depicts a transilluminator 110 having a high intensity light source 120 for illuminating/enhanced visualization of a nucleic acid specimen 122 (inclusive of the underlying tray). The transilluminator 110 is depicted in combination with: (i) the nucleic acid specimen 122, (ii) an electrophoresis chamber 114, (iii) a camera/video recording device 118 disposed over and viewing the specimen 122 (i.e., internally of the chamber 114), and (iv) a power source 130 for energizing the electrophoresis chamber 114, the camera/video recording device 118, and a high intensity light source 120.

The light source 120 is adapted to emit high intensity white light through a filtering plate 116 interposed between the light source 120 and the nucleic acid specimen 122. The plate 116 filters all wavelengths of light below about 430 nanometers and above about 500 nanometers, therefore, allowing light energy in the relatively narrow band therebetween (i.e., light in the visible violet/blue light spectrum). A switch 132 controls the supply of power to the light source 120 from the power supply 130.

It will be appreciated that, to obtain light of sufficient intensity in a narrow band, an underlying white light source 120 will necessarily produce vastly greater amounts of light energy (and, consequently, heat) which must be filtered/absorbed. As a result, the filter plate 116 is a costly component of the transilluminator 110.

To protect the glass plate 116, it is common to employ heavy gauge steel box construction to prevent flexure and cracking of the glass during use. Furthermore, cooling fans (not shown) are commonly employed to convection cool the glass filter 116.

Additionally, it may be desirable to illuminate the nucleic acid specimen with various wavelengths of light, (e.g., 250 vs. 300 nanometers/300 vs., 350 nanometers etc.), to compare and contrast any observed differences. Hence, it is common to employ more than one transilluminator, each equipped with a different wavelength glass filter, or a single transilluminator having replaceable or interchangeable filter plates 116.

From the foregoing it will be apparent that conventional transilluminators, such as the one depicted/described in the Smoot et al. '655 patent, are heavy to manipulate and costly to fabricate. Furthermore, such transilluminators consume large quantities of power (i.e., as a consequence of the generation and requirement to dispose of the heat produced). Moreover, and perhaps most importantly, prior art transilluminators do not adequately solve or address the need/desirability to generate multiple bands of light for the purpose of comparing/contrasting infinitesimally small nucleic acid fragments (some as small as two nanograms, 2×10⁻⁹ gms in size).

Other patents which discuss/describe transilluminators, transilluminators for electrophoresis devices, and/or apparatus for enhancing the illumination of objects/specimens for visual examination include Saravis U.S. Pat. No. 3,764,513, Golias U.S. Pat. No. 4,360,418, and Johannsen et al. U.S. Pat. No. 5,736,744.

A need, therefore, exists for a transilluminator which is light-weight, low cost, and energy efficient. Furthermore, a need exists for such a transilluminator which may be adapted to generate multiple wavelengths of light for enhancing the analysis/examination of nucleic acid specimens/fragments.

### SUMMARY OF THE INVENTION

A transilluminator is provided for enhanced visualization of an object/specimen during its examination and/or analysis such as nucleic acid fragment in an electrophoresis apparatus/process. In the described embodiment, the transilluminator includes a transmittance plate adapted to support the nucleic acid fragment/specimen, and a light source adapted to project an array of light through the plate for illumination of the nucleic acid specimen. Light Emitting Diodes (LEDs) are preferably juxtaposed in a substantially planar array such that the LEDs illuminate the plate with substantially uniform intensity. The transmittance plate, LEDs or a combination thereof filters and/or produces light energy within a predefined spectrum of light energy. In the preferred embodiment, the illuminating light is in the ultraviolet or near-ultraviolet light spectrums to stimulate a fluorescent response in nucleic acid fragments/specimens. Furthermore, two or more LEDs which vary in wavelength emission may be employed for the purpose of varying the illumination characteristics of the transilluminator.

Other objects, aspects and advantages of the present invention will become apparent to those skilled in the art upon reading the following detailed description, when considered in conjunction with the appended claims and the accompanying drawings briefly described below.

### BRIEF DESCRIPTION OF THE DRAWING

For the purpose of illustrating the invention, there is shown in the drawings various forms that are presently preferred; it being understood, however, that this invention is not limited to the precise arrangements and constructions particularly shown.

FIG. 1 is an exploded view of a prior art electrophoretic device having a transilluminator employing a high intensity light source projecting randomly dispersed light energy through a filter for illuminating a nucleic acid specimen/fragment.

FIG. 2 is an isolated perspective view of an exemplary embodiment of a transilluminator according to the present invention.

FIG. 3 is an exploded isometric view of the inventive transilluminator illustrated in FIG. 2 including a housing, a transmittance plate, an array of Light Emitting Diodes (LEDs) disposed in combination with the base of the housing and a cover pivotally mounted to the housing.

FIG. 4 is a top view of the transilluminator including a nucleic acid fragment/specimen disposed upon and supported by the transmittance plate for illumination and examination.

FIG. 5 is a cross-sectional view taken substantially along line 5-5 of FIG. 4.

FIG. 6 is an enlarged, broken-away sectional view of the LED light source wherein collimated rays of light illuminate the nucleic acid fragment/specimen.

FIGS. 7a and 7b depicts an alternate embodiment of the invention wherein the array of LEDs comprises at least two interspersed LEDs, one of the interspersed LEDs emitting light within a first light spectrum (FIG. 7a) and another of the interspersed LEDs emitting light within a second light spectrum (FIG. 7b) so that a nucleic acid fragment/specimen may be compared and contrasted at different wavelength emissions.

FIG. 8 is a cross-sectional view of a transilluminator illustrating a preferred mounting arrangement for a diffusion screen.

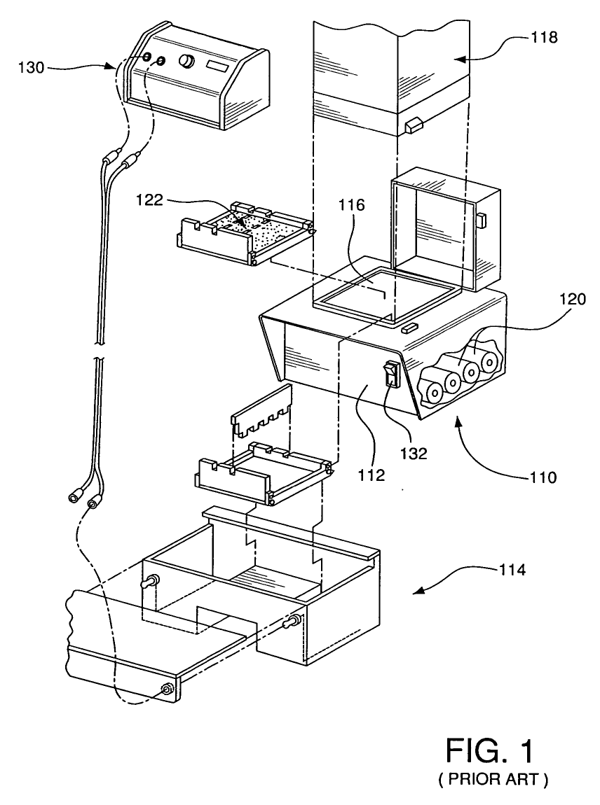

### DETAILED DESCRIPTION OF THE DRAWINGS

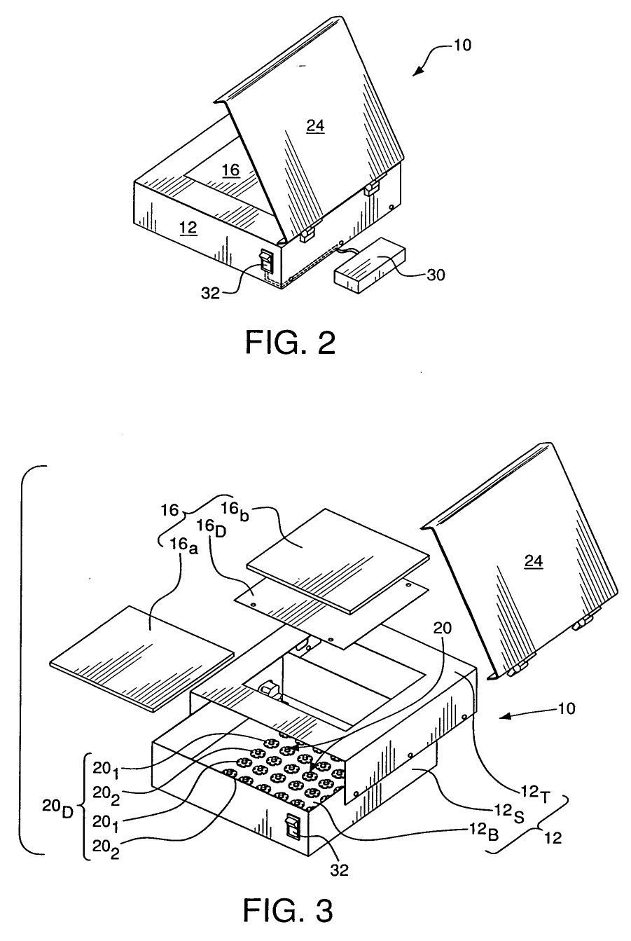

Referring now to the drawings wherein like reference numerals identify like elements, components, subassemblies etc., FIG. 2 depicts an exemplary embodiment of a transilluminator 10 according to the teachings of the present invention. While the transilluminator 10 will be described in the context of a small portable unit, i.e., resembling a briefcase in appearance, it should be appreciated that the transilluminator 10 may vary in size and shape, whether mobile or stationary. Furthermore, while the exemplary embodiment described herein is directed to illuminating nucleic acid fragments/specimens, the transilluminator 10 is useful for illuminating any object requiring enhanced visualization.

In the broadest sense of the invention, and referring to FIGS. 2 and 3, the transilluminator comprises a transmittance plate or layer 16 adapted to support an object/specimen (not shown), and a light source 20 adapted to project an array of light through the plate 16 to illuminate the object/specimen. The array of light may be substantially collimated. As used herein, the “transmittance plate” covers any layer of material that permits at least some light to pass though, whether by absorption, reflection, diffusion, refraction, and includes glass and plastic materials. Furthermore, the term expressly covers transparent or clear material, as well as colored glass or plastic. Also, the plate need not be planar since it is contemplated that the plate may be shaped in various ways. It is also contemplated that the transmittance plate 16 may be a flexible film or coating. In the terms of a coating, the transmittance layer would be applied to another support substrate. The term “specimen” is intended to include any supporting tray or other convenient means/method for handling, transporting and/or supporting the object to be examined.

The transilluminator 10 includes a housing 12 having a base, a top and sidewall portions, 12_{B}, 12_{T} and 12_{S}, respectively. As those skilled in the art understand, any suitable shaped housing can be used in the present invention. The housing 12 can be made from any suitable material, such as a thermoplastic, a thermoset composite, or a metallic material. Preferably, the base and sidewalls 12_{B}, 12_{S} define an enclosure which is substantially impervious to light, i.e., opaque. The transmittance plate 16 is disposed in combination with the top portion 12_{T} of the housing 12 and may comprise multiple layers 16a, 16b and 16_{D} to yield the desired illumination characteristics (discussed in greater detail when describing the operation and function of the plate 16). The transmittance plate 16 can be separate from the housing 12 or may be hinged to the housing.

A cover 24 may be employed to protect an operator from potentially harmful electromagnetic radiation, e.g., ultraviolet radiation, when, and/or if, wavelengths in these spectrums are employed. Alternatively or additionally, the cover may assist in providing color contrast and/or color cancellation (of visible light) to enhance the visualization of the specimen. For example, a blue LED can tend to emit a combination of yellow and blue light. If the cover is made from yellow colored glass or plastic, it filters out the blue so that the yellow light is enhanced. Thus, the cover can be selected to provide beneficial contrast between the background and the emitted light. In one embodiment, the cover is made from plastic material that has an amber color. The cover can be removable form the housing 12 or may be hinged to the housing for ease of accessing the top of the housing.

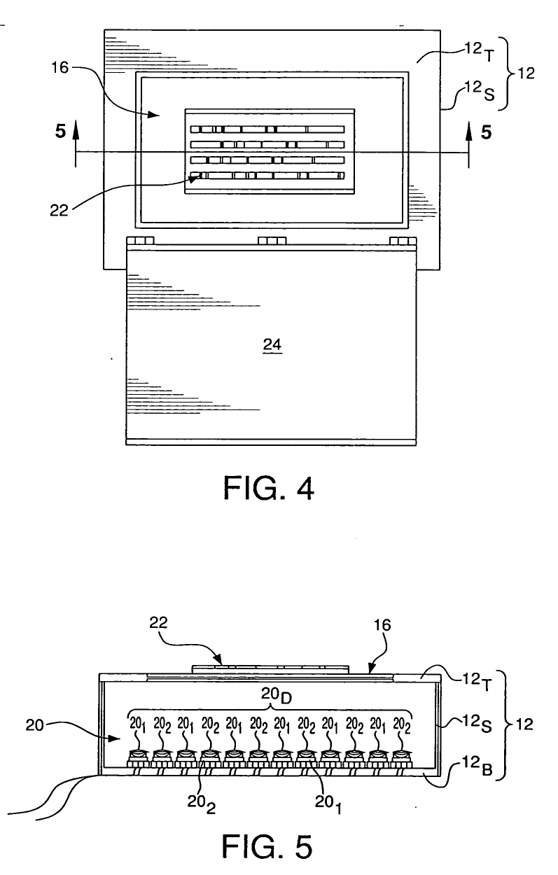

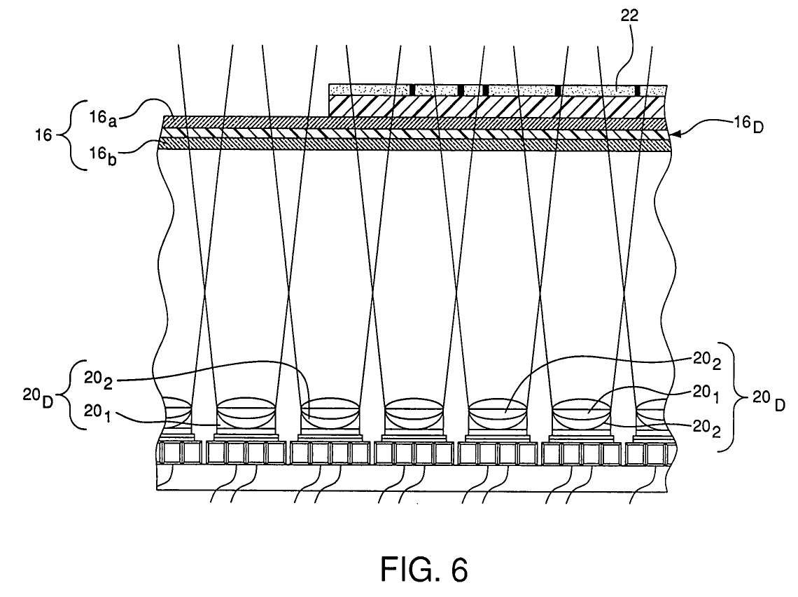

In FIGS. 4, 5 and 6 the light source 20 may comprise a plurality of Light Emitting Diodes (LEDs) 20_{D}, disposed in combination with the base 12_{B} and arranged in a preferably substantially planar array. More specifically, the LEDs 20_{D} (see FIGS. 5 and 6) are adapted to emit light which is directed upwardly through the transmittance plate 16. A power source 30 supplies electric power to the array of LEDs 20_{D} and may be turned on or off by means of a conventional switch 32, such as a rocker switch, disposed in a sidewall 12_{S} or top 12_{T} portion of the housing 12.

As discussed in the Background of the Invention, nucleic acid fragments/specimens are best observed and examined in the presence of light energy within a predefined light spectrum (e.g., a light spectrum which maximizes the contrast for viewing or produces a fluorescent response when illuminating the object/specimen). With respect to the latter, fragments/specimens 22, which are stained with certain materials, become visible and/or produce a fluorescent response in the presence of light energy in a predefined light spectrum. One such material, commonly used in electrophoresis, is ethidium bromide which fluoresces in the presence of ultraviolet and/or near-ultraviolet light. In the context used herein, the term “ultraviolet light” means light energy having a wavelength between about 250 nanometers to about 400 nanometers and “near-ultraviolet” means light energy having a wavelength between about 400 nanometers and about 500 nanometers.

Referring to FIGS. 5 and 6, high or low intensity LEDs 20^{D} may be employed depending upon the preparation of the nucleic acid fragment/specimen 22, e.g., whether or not the specimen 22 is stained with ethidium bromide. By “high intensity” is meant that the LEDs are capable of at least one (1) watt of power dissipation from the LED. The LEDs 20^{D} in the present invention are preferably designed to provide at least one (1) watt of power dissipation. Such LEDs 20_{D} typically include an emitter mounted to a base. LEDs 20_{D} useful for the practicing the invention are available from Lumileds located in San Jose, Calif. under the trade name Luxeon™ Power Light Sources.

“Low intensity”, by comparison, means LEDs 20_{D} which consume/generate less than one (1) watt of light energy. Such low intensity LEDs are available from many sources, including Cree Semiconductors located in Durham, N.C. In order to provide the desired illumination, it may be necessary to increase the number of low intensity LEDs.

The transilluminator 10 of the present invention contemplates a variety of structural combinations to produce the requisite visual enhancement sought for examining objects and specimens, especially objects or specimens as delicate and challenging as a nucleic acid fragment. More specifically, the transmittance plate 16, the light source 20, individual LEDs 20_{D}, or a combination thereof may be configured to filter and/or produce light energy within a predefined spectrum of light energy. For example, in one embodiment of the invention, high intensity LEDs 20_{D} emitting visible light, such as “white” light (which is visible light in all light spectrums) in combination with a filtering transmittance plate 16 may be employed to improve the thermal or heat transfer properties of the transilluminator 10. That is, the LEDs 20_{D} specified herein provide high intensity/luminous flux yet consume only a fraction of the power of conventional prior art white transilluminator bulbs. Consequently, the transilluminator 10 eliminates the requirement for heavy steel construction, cooling fans and/or an elaborate network of cooling fins/thermal paths for creating heat sinks.

In another embodiment, the transmittance plate 16 may be non-filtering (i.e., allowing the passage of all wavelengths of light energy), however, the light source 20 is adapted to produce the requisite illumination/light characteristics. That is, in this embodiment, the light source 20 may be selected to produce a uniform intensity of illuminating light within a predefined spectrum. For example, the transmittance plate 16 may serve no other purpose than to support a nucleic acid fragment/specimen, while the LEDs collectively produce emissions within a particular wavelength band, e.g., violet/purple light from about 430 nanometers to about 500 nanometers in wavelength. The LEDs 20_{D} emitting light within such violet/purple light spectrum, consume even less energy than the white light LEDs described in the preceding paragraph, hence, the thermal attributes of the transilluminator 10 are yet further enhanced.

In yet another embodiment, the transmittance plate 16 may be reconfigurable from, for example, non-filtering to filtering while the light source 20 is selected to produce light within a predefined light spectrum (similar to the embodiment immediately preceding). As such, the transilluminator 10 may irradiate the nucleic acid fragment/specimen 22 with two or more wavelengths of light, e.g., in the visible light spectrum and ultraviolet or near-ultraviolet light spectrum, for the purpose of comparing and contrasting specimen results. One example of visible light that can be used in the present invention is a combination of red, blue and green LEDs which emit light that appears white. In this embodiment, the transmittance plate 16 may include lower and upper plates 16a, 16b (see FIGS. 3 and 6). The lower plate 16a may comprise a substantially clear or white plate, such as one made from glass or acrylic material, which provides minimum filtering. The lower plate 16a may be fixed to or movable/removable from the housing. The upper plate 16b comprises a separate filter, such as a blue filter. (Although the term “plate” is used to refer to the upper filter, it should be recognized that the filter may made from flexible material, such as plastic film, that provides that necessary filtering. If the upper filter is flexible, it could be supported by the lower plate 16b.) The upper plate 16b is movable/removable so that it may be placed upon or substituted for the clear or white acrylic lower plate 16a. In this embodiment, white LEDs 20 are used to provide the illumination.

During operation of the transilluminator in this embodiment of the invention, when white light is needed for illumination/examination, the LED array produces the requisite light energy and the white or clear lower plate 16a allows the light to pass essentially unaffected. The upper blue filter 16b is not used. When filtered light is needed for illumination/examination of a nucleic acid fragment/specimen 22, the upper filter plate 16b is placed on, under or substituted for the lower plate 16a. The upper filter plate 16b may be hingedly attached to the housing so that it can simply and easily be moved into position over the lower plate 16a when needed. It is also contemplated that multiple upper filtering plates 16b may be used, each plate filtering different wavelengths of light.

In a variation on the above embodiment, the upper plate 16b is a substantially clear or white plate, which provides minimum filtering. The upper plate 16b is attached to or placed on the housing and designed to support the sample being examined. The lower plate 16b is a movable/removable filter, such as a blue filter. The lower plate 16a may be slidable into the housing through a slot. The lower plate seals against the housing to prevent leakage.

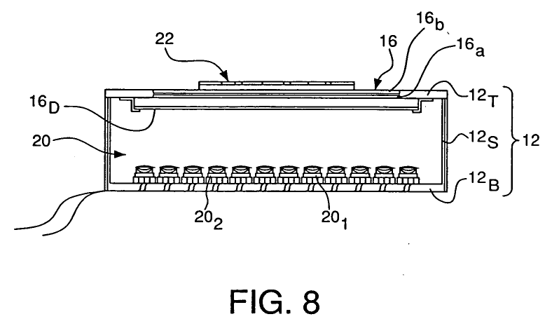

In one preferred embodiment, a sheet 16_{D} of diffusing material is located below the lower and upper plates 16a, 16b to evenly disperse the light energy. This embodiment is generally shown in FIG. 8 where the diffusing screen is mounted on a frame which, in turn, it mounted to the top of the housing. While the diffusing/dispersing sheet is preferably located closest to the LEDs, it should be readily apparent that the sheet can be placed at any suitable location. The diffusing sheet or screen 16_{D} may be made from a polymer material. Diffusing screen may be removable. Diffusing screens are well known in the field of lighting for use in transilluminators and, thus, no further description is necessary.

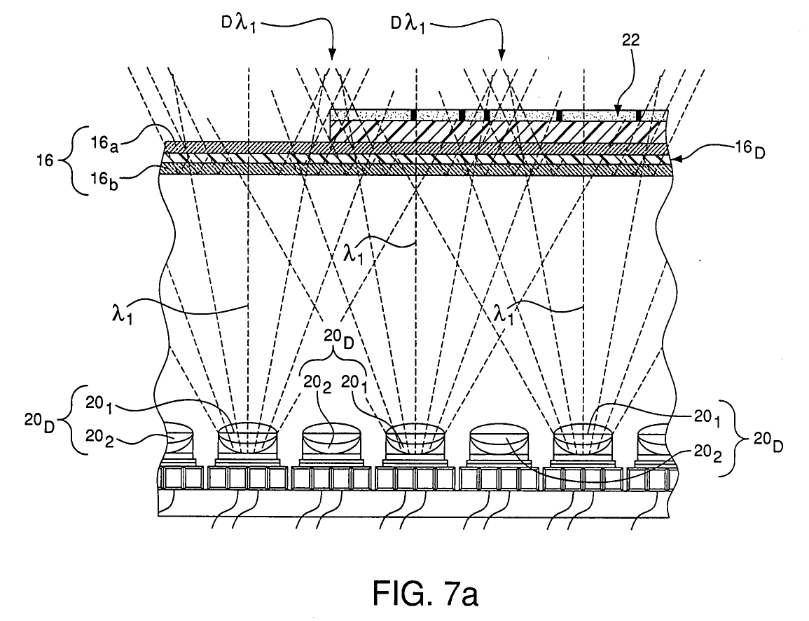

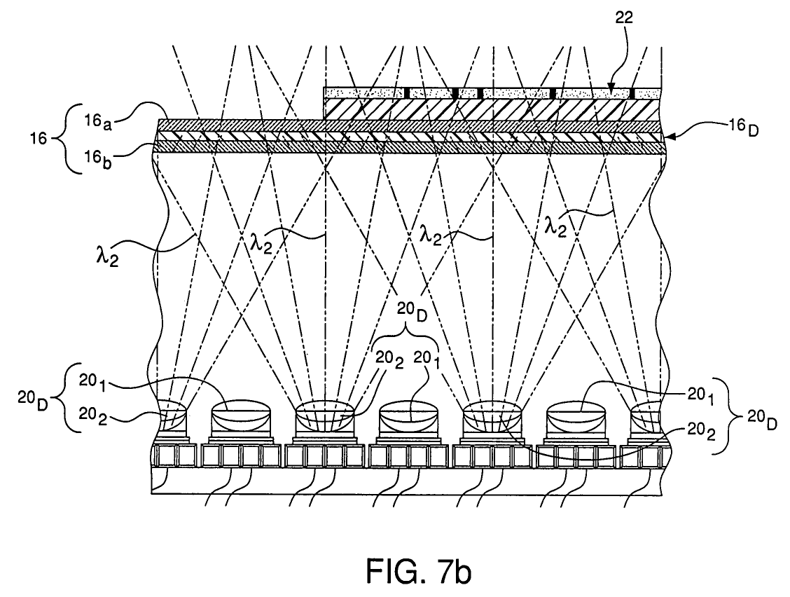

In yet another embodiment of the invention, the transilluminator 10 may illuminate the nucleic acid fragment/specimen 22 with two or more wavelengths of light by employing LEDs 20_{D} which differ in wavelength emission. In FIGS. 7a and 7b, LEDs 20₁ having a characteristic first wavelength emission XI, i.e., emitting light within a first spectrum of light energy, may be interspersed with other LEDs 202 having a characteristic second wavelength emission 2. Specifically, the LEDs (e.g., LED 201) with the same characteristic wavelength are preferably interspersed with the other LEDs (e.g., LED 202). Alternately, certain of the LEDs may be grouped in a predetermined spatial arrangement. The location and positioning of the LEDs is preferably set so as to provide uniform illumination. For example, it is contemplated that the transilluminator in this embodiment would include a plurality of LEDs that emit visible light, such as yellow, red, or white (wherein “white” light can be created by activating red, blue, and green LEDs together) and a plurality of blue or UV LEDs. It should be readily apparent that the array of LEDs may, in one embodiment, comprise groups of red, blue, and green LEDs at each location. The LEDs would be wired such that when general viewing is desired, all the LEDs are activated. When inspection of a specimen is desired, only the blue LEDs are activated.

While the array of LEDs has been described as including LEDs that emit different colors being interspersed, it is also contemplated that LEDs that emit light within one spectrum can be mounted on one side of the housing and LEDs that emit light within another spectrum would be mounted on the other side. For example, UV LEDs might be mounted on one side of the housing and white LEDs on the other. In this embodiment, it is preferable that the transmittance plate be designed to enhance the desired illumination. Thus, the transmittance plate may be half opal glass and half colored glass, the visible light LEDs under the opal glass and the UV LEDs under the colored glass.

The transilluminator switch 32 (shown in FIGS. 2 and 3) in this embodiment may be electrically connected to the LEDs 20_{D} so as to selectively energize only those LEDs having a common wavelength characteristic. That is, during one operation or examination procedure, LEDs 20₁ emitting, for example, ultraviolet light emissions (e.g., λ₁≈250-400 nanometers) are energized while, during a second operation or procedure, the LEDs 202 emitting near-ultraviolet emissions (e.g., λ₂≈400-500 nanometers) or visible light, such as white, yellow, green, or red light emissions, are energized. Consequently, the nucleic acid fragment/specimen 22 may be analyzed by a single transilluminator 10 without the requirement to remove, reset, and re-examine the specimen 22. It will be recalled that, in the prior art, this operation typically required multiple transilluminators each having emissions of a dedicated wavelength.

In summary, the teachings of the present invention provide a low cost, energy efficient transilluminator 10 for enhanced visualization of objects/specimens undergoing examination and/or analysis. The array of LEDs 20_{D} results in low energy consumption i.e., high luminous efficiency, which significantly reduces the heat transfer requirements of the transilluminator. As such, cooling fans, thin fins, and/or other heat dissipating devices/structures are eliminated, and the requirement for such devices substantially mitigated. In the context of an electrophoresis device, the LED transilluminator is particularly effective inasmuch as the energy and heat dissipation requirements thereof are perhaps the most challenging of all transilluminator applications.

The transmittance plate 16, the array of LEDs 20, individual LEDs 20_{D}, 20₁, 20₂ and/or a combination thereof may be adapted to filter and/or produce light energy within a predefined spectrum of light energy. In addition to reducing the ongoing cost of maintenance, the transilluminator 10 of the present invention reduces the initial set-up and capital investment required to perform optical densitometry. That is, the transilluminator 10 can potentially perform the functions of multiple transilluminators by selectively energizing specific LEDs within the array, hence, the capital investment required to perform such functions may be significantly or dramatically reduced.

It will be appreciated that other modifications, additions, deletions and/or omissions may be made to the teachings of the invention without departing from the spirit or essential attributes thereof and, accordingly, reference should be made to the appended claims, rather than to the foregoing specification, as indicating the scope of the invention.

We claim:

1. A transilluminator for visually examining a specimen, comprising:
a housing having a top;
an array of light emitting diodes located within the housing so as to emit light when activated; and
a transmittance plate located between the light emitting diodes and the top, the plate adapted to support a specimen.

2. The transilluminator according to claim 1 wherein the array of LEDs emit light within a predefined spectrum of light.

3. The transilluminator according to claim 1 wherein the transmittance plate filters the light emissions of the LEDs to permit transmission of light within a predefined light spectrum (wavelength).

4. The transilluminator according to claim 2 wherein the predefined light spectrum is selected to effect a fluorescent response in the specimen.

5. The transilluminator according to claim 3 wherein the predefined light spectrum is in the ultraviolet light spectrum.

6. The transilluminator according to claim 3 wherein the transmittance plate is adapted to disperse the light emissions of the LEDs in a substantially uniform pattern.

7. The transilluminator according to claim 6 wherein the transmittance plate includes a diffusive media for dispersion of the LED light emissions.

8. The transilluminator according to claim 7 wherein the diffusive media is a sheet material located between the transmittance plate and the array of LEDs.

9. The transilluminator according to claim 2 wherein the array of LEDs includes at least two groups of LEDs, each group of LEDs emitting light within a different light spectrum than the other groups of LEDs, and further comprising a switching device for selectively energizing each of the groups of LEDs.

10. The transilluminator according to claim 9 wherein the LEDs are uniformly interspersed.

11. The transilluminator according to claim 1 wherein the base is adapted to transfer heat away from the LED array, and the top portion including a cover plate.

12. A transilluminator for visually examining a specimen, comprising:
a housing having a top;
a transmittance plate on or in the housing for supporting the specimen;
an array of light emitting diodes located within the housing and adapted to emit light to illuminate a specimen;
the plate adapted to filter the light emissions of the LEDs to transmit light within a predefined light spectrum.

13. The transilluminator according to claim 12 wherein the predefined light spectrum is in the ultraviolet light spectrum.

14. The transilluminator according to claim 13 wherein the transmittance plate includes a diffusive media for dispersion of the LED light emissions.

15. The transilluminator according to claim 14 wherein the diffusive media is a sheet material interposed between the transmittance plate and the array of LEDs.

16. A transilluminator for visually examining a specimen, comprising:
a housing having a top portion,
a transmittance plate located at or near the top portion of the housing and adapted to support a specimen, and
an array of light emitting diodes located within the housing and adapted to emit light to illuminate the specimen, the array of LEDs for emitting light within a predefined light spectrum.

17. The transilluminator according to claim 16 wherein the predefined light spectrum is in the ultraviolet light spectrum.

18. The transilluminator according to claim 16 wherein the array of LEDs includes at least two groups of LEDs, each group of LEDs emitting light within a different light spectrum than the other groups of LEDs, and further comprising a switch for selectively energizing each of the groups of LEDs.

19. The transilluminator according to claim 18 wherein the light spectrum emitted by at least one of the groups of LEDs is within the ultraviolet light spectrum.

20. The transilluminator according to claim 19 wherein the LEDs are uniformly interspersed.

21. The transilluminator according to claim 16 wherein the transmittance plate filters the light emissions of the LEDs to transmit light within the ultraviolet light spectrum.

22. The transilluminator according to claim 19 wherein the transmittance plate is adapted to disperse the light emissions of the LEDs in a substantially uniform pattern.

23. The transilluminator according to claim 22 wherein the transmittance plate includes a diffusive media for dispersion of the LED light emissions.

24. The transilluminator according to claim 22 wherein the diffusive media is a sheet material interposed between the transmittance plate and the array of LEDs.

25. The transilluminator according to claim 9 wherein one of the groups of LEDs emits light in the ultraviolet light spectrum and the other group of LEDs emits light in the visible light spectrum.

26. The transilluminator according to claim 25 wherein the visible light spectrum provides substantially white light emission.

27. The transilluminator according to claim 11 wherein the cover plate is hinged to the housing.

28. The transilluminator according to claim 12 wherein the array of LEDs includes at least two groups of LEDs, each group of LEDs emitting light within a different light spectrum than the other groups of LEDs, and further comprising a switching device for selectively energizing each of the groups of LEDs.

29. The transilluminator according to claim 28 wherein one of the groups of LEDs emits light in the ultraviolet light spectrum and the other group of LEDs emits light in the visible light spectrum.

30. The transilluminator according to claim 29 wherein the visible light spectrum provides substantially white light emission.

31. The transilluminator according to claim 19 wherein the light spectrum emitted by the other group of LEDs is within the visible light spectrum.

32. The transilluminator according to claim 31 wherein the visible light spectrum provides substantially white light emission.
